(19) United States
(12) Patent Application Publication
(10) Pub. No.: US 2022/0257411 A1
(43) Pub. Date: Aug. 18, 2022

(54) COLD AIR THERAPY DEVICE, METHOD OF APPLYING A COOLED AIR FLOW AND USE OF AN AIR DISINFECTION DEVICE

(71) Applicant: Zimmer MedizinSysteme GmbH, Neu-Ulm (DE)

(72) Inventor: Michael Zimmer, Neu-Ulm (DE)

(21) Appl. No.: 17/631,278

(22) PCT Filed: May 27, 2020
(86) PCT No.: PCT/EP2020/064681

§ 371 Date: Jan. 28, 2022

(30) Foreign Application Priority Data
Jul. 29, 2019 (DE) 10 2019 211 247.1

(51) Int. Cl. A61F 7/00; A61L 9/20

(52) U.S. Cl. CPC A61F 7/0085; A61L 2209/12; A61F 2007/0064; A61L 9/20

(57) ABSTRACT

The present invention provides a cold air therapy device (1) for applying a cooled air flow to a body surface, comprising: cooling means (2) configured to cool the air flow to be applied to the body surface; air guiding means (3) coupled to the cooling means (2) and configured to direct the air flow, in order to direct the air flow to be applied to the body surface, which is cooled by the cooling device (2), to a cold air outlet (4); and an air disinfection device (5) which is configured to at least reduce the germ load and/or the bacterial load of the air flow to be applied to the body surface.

The present invention relates to a cold air therapy device comprising an air disinfection device, a method of applying a cooled air flow as well as a use of an air disinfection device.

For many applications in the medical field, cold air therapy devices are nowadays used for cooling of body regions. In this respect, air is blown by means of a fan through a cold reservoir whereby the air is cooled down. The cooled air is applied to the patient's body by means of a hose or an appropriate air duct. The air to be cooled may, for example, be taken from an appropriate sterile air reservoir. This, however, is expensive and has the disadvantage that the air reservoir needs to be refilled over and over again. Alternatively, ambient air could therefore also be used. However, if germs, bacteria or the like are present in the air or in the supply line, there is a risk that they will be applied to the patient by the air flow. This should be avoided as far as possible, particularly when treating body surfaces showing open wounds.

Document DE 32 42 881 A1 discloses a device for generating a cold gas flow by which device a cold, non-aggressive liquid gas is sprayed and vaporized in a non-aggressive carrier gas flow so that a cold gas flow of defined flow rate and temperature is available upstream.

In view of this background, it is an object according to the present invention to provide an improved cold air therapy device which provides a reduced risk of infection.

According to the present invention, this object is achieved by a cold air therapy device comprising the features of claim 1, a method of applying a cooled air flow comprising the features of claim 14, and a use of an air disinfection device comprising the features of claim 15.

Accordingly, a cold air therapy device is provided for applying a cooled air flow to a body surface. The cold air therapy device comprises: a cooling device which is configured to cool the air flow to be applied to the body surface; an air guiding device which is coupled to the cooling device and which is configured to direct the air flow, which is cooled by the cooling device and which is to be applied to the body surface, to a cold air outlet; and an air disinfection device which is configured to at least reduce the germ load and/or the bacterial load of the air flow to be applied to the body surface.

Furthermore, a method is provided for applying a cooled air flow to a body surface. The air flow to be applied to the body surface is cooled, the cooled air flow to be applied to the body surface is directed to the body surface, and the germ load and/or the bacterial load of the air flow to be applied to the body surface is reduced.

Furthermore, use of an air disinfection device is provided for reducing the germ load and/or the bacterial load of an air flow of a cold air therapy device to be applied to a body surface before its application to the body surface.

The present invention is based on the principle of disinfecting an air flow on its way to application to a body surface to be cooled. Accordingly, normal ambient air may also be used as a coolant without creating a possible risk of infection. Compared to a cold air therapy device having a special air reservoir, the cold air therapy device according to the present invention may be manufactured at low cost and may be used in a more flexible way. Due to the compact configuration with fewer different components, performance of service and maintenance work is advantageously simplified.

Thus, the germ load and/or the bacterial load should be reduced at least significantly and ideally as completely as possible. For example, the germ load and/or the bacterial load may be reduced by at least 50%, advantageously by at least 90% and, preferably, by at least 99%.

A vast variety of methods is conceivable for performing disinfection, for example, directing the air flow to be applied to the body surface through a chemical disinfectant such as hydrogen peroxide, chlorine, ozone, alcohol, or the like. Disinfection methods based on electromagnetic radiation, heat supply or plasma may also be used. Each method of disinfection shows its specific advantages and disadvantages.

The germ load and/or the bacterial load are usually distributed homogeneously in the entire air flow to be applied to the body surface. Therefore, the entire air flow to be disinfected by the air disinfection device should also be disinfected as uniformly as possible in order to prevent parts of the air flow to be applied to the body surface from possibly being insufficiently disinfected. This must be taken into account when designing the air disinfection device.

Advantageous embodiments and further configurations result from the dependent claims and from the description when taken in conjunction with the Figures.

According to a further embodiment, the air disinfection device may comprise at least one UV light source. UV light refers to electromagnetic radiation in the wavelength range between 100 nm and 380 nm, which has a germicidal effect, particularly in the wavelength range between 100 nm and 280 nm, and is therefore suitable for reducing the germ load and/or bacterial contamination of air which is irradiated with such UV light, at the same time being used easily and safely advantageously when compared to, for example, chemical or radioactive disinfection methods.

According to a further embodiment, the air disinfection device may comprise a housing and may be arranged in such a way that the cooled air flow passes through the housing during operation, wherein the UV light source is positioned inside the housing. By means of a housing which is suitably provided for this purpose, the air disinfection device may be configured in such a way that the air flow through the air disinfection device is as uniform as possible. This prevents insufficiently disinfected air from being applied to the body surface.

In accordance with a further embodiment, the housing of the air disinfection device may comprise a funnel-shaped outlet region. Such an outlet region reduces the occurrence of eddy currents when the air flow exits the housing. This allows the airflow to be irradiated with UV light in a uniform manner, which prevents insufficiently disinfected air from being applied to the body surface.

According to another embodiment, the at least one UV light source may be positioned in a central area of the housing of the air disinfection device with respect to a flow direction of the cooled air flow. In this configuration, the air flow to be disinfected may flow around the at least one UV light source, preferably in laminar form. This reduces the impact of the UV light source on the air flow in an advantageous manner and ensures uniform disinfection of the air flow.

According to another embodiment, the air disinfection device may comprise a tube of transparent material through which the air flow to be applied to the body surface flows. The at least one UV light source may be positioned outside the tube. The separation of the at least one UV light source from the air flow to be disinfected prevents the UV light source from adversely affecting the air flow. The material used for the tube should be transparent in the wavelength range of UV light, wherein possible materials include glass or plastic.

According to another embodiment, the air disinfection device may comprise a multitude of UV light sources. This may advantageously increase the radiation power acting on the air flowing through the air disinfection device, which in turn increases the degree of reduction of the germ load and/or the bacterial load of the air flow to be applied to the body surface.

According to another embodiment, UV light sources may be arranged in a ring shape. This configuration has proven to be particularly advantageous, as the volume fraction of the air flow illuminated by the UV light sources is particularly high in this case, without causing unfavorable turbulences in the air flow to be disinfected.

According to another embodiment, the at least one UV light source may be arranged in such a way that the air flow to be applied to the body surface flows through the air disinfection device along a meandering or spiral path. This increases the dwell time of the air to be disinfected in the air disinfection device. During this time the UV radiation acts on the air flow and the higher radiation dose absorbed further reduces the germ load and/or the bacterial load of the air flow.

According to another embodiment, at least one interior surface of the housing may be covered or coated with a UV light reflecting material, preferably aluminum. For example, at least one interior surface of the housing may be covered with aluminum foil. Alternatively, the housing may also be made of aluminum or covered with, for example, polytetrafluorethylene or polycarbonate. This causes UV light to be reflected from the housing wall back into the airflow. The intensity of the UV light acting on the airflow is thus advantageously increased, which further reduces the germ load and/or bacterial load of the airflow to be applied to the body surface.

According to another embodiment, the air disinfection device may be positioned at the cold air outlet. In particular, the air disinfection device may be integral with the cold air outlet. In this configuration, the air flow to be applied to the body surface is disinfected at the last moment before being applied to the body surface. This is an advantageous way to avoid that the air flow is again exposed to a germ load on its way from the air disinfection device to the application location.

An air filter may be provided in accordance with a further embodiment. The filter is preferably arranged in the direction of the air flow behind the air disinfection device, but may also be arranged in the direction of the air flow in front of the air disinfection device or at another location in the air flow line. In this way the germ load of the air flow may be further reduced. Other inorganic dirt particles may also be filtered with an air filter of this type, which is also highly desirable upon application to body surfaces, particularly in the case of open wounds.

According to another embodiment, a ventilation device may be provided which is configured to generate the air flow to be applied to the body surface. The ventilation device allows the cold air therapy device to be used independently by means of the integral ventilation device without having to rely on other devices such as external ventilation systems.

The above embodiments and configurations may be combined with each other as desired, if it is sensible. Further possible configurations, further embodiments and implementations according to the present invention also include combinations of features according to the present invention described above or below with regard to the exemplary embodiments which are not explicitly mentioned. In particular, the skilled person may also add individual aspects as improvements or additions to the respective basic form according to the present invention.

The present invention will be explained in more detail below in conjunction with the embodiments presented in the schematic figures, wherein:

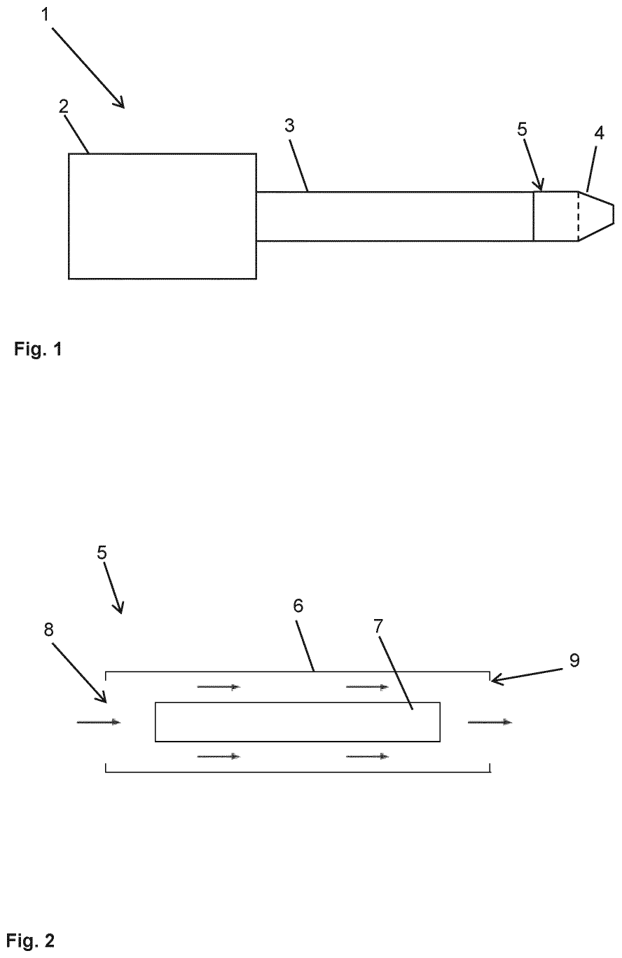

FIG. 1 is a schematic side view of a cold air therapy device according to an exemplary embodiment of the present invention;

FIG. 2 is a schematic sectional view of an air disinfection device for a cold air therapy device according to an exemplary embodiment of the present invention;

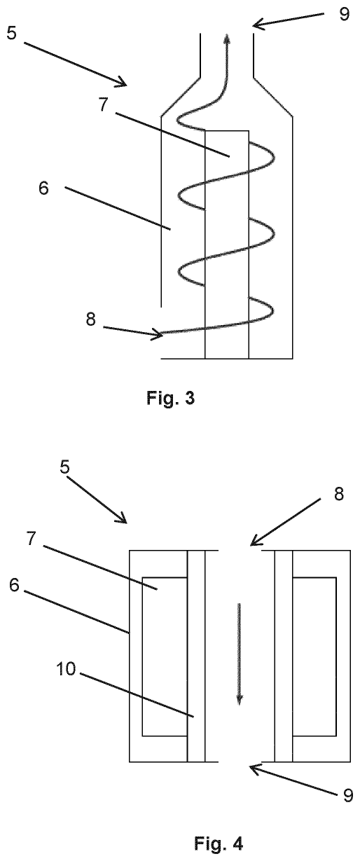

FIG. 3 is a schematic sectional view of an air disinfection device for a cold air therapy device according to an exemplary embodiment of the present invention;

FIG. 4 is a schematic sectional view of an air disinfection device for a cold air therapy device according to an exemplary embodiment of the present invention;

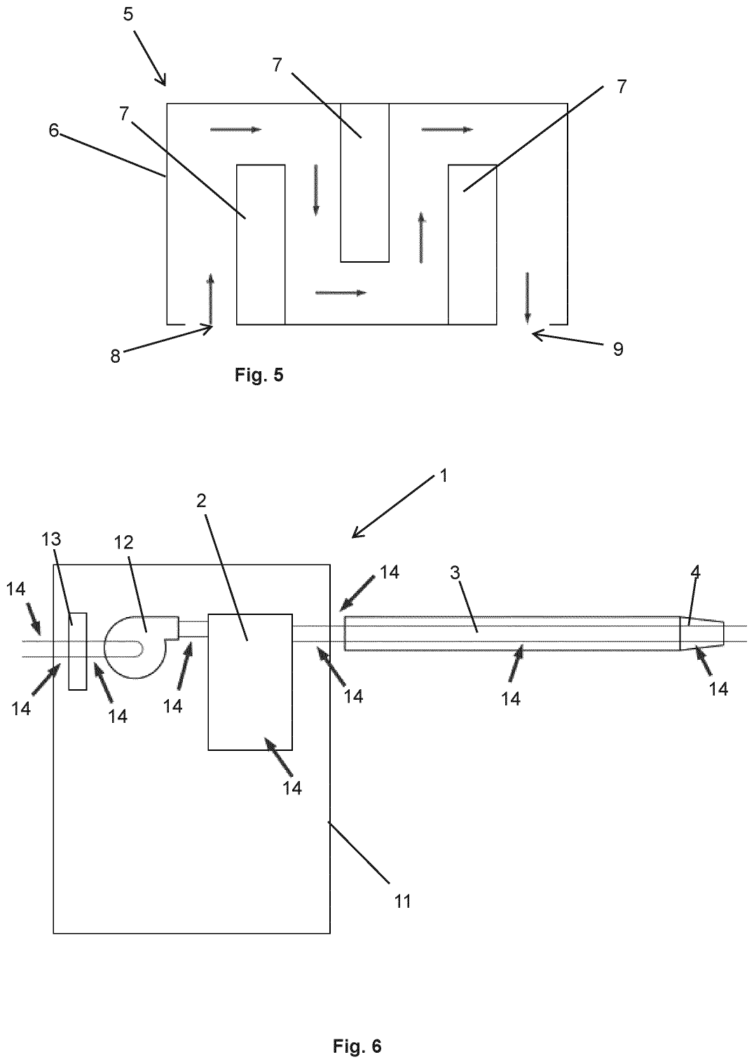

FIG. 5 is a schematic sectional view of an air disinfection device for a cold air therapy device according to an exemplary embodiment of the present invention;

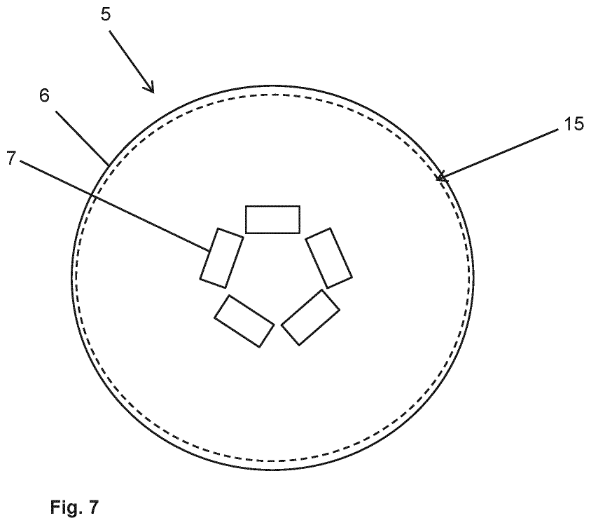

FIG. 6 is a schematic side view of a cold air therapy device according to a further exemplary embodiment of the present invention; and

FIG. 7 is a schematic cross-sectional view of an air disinfection device for the cold air therapy device shown in FIG. 7

The enclosed Figures are intended to provide a better understanding of the embodiments according to the present invention. The Figures illustrate embodiments and serve in connection with the description to explain the principles and concepts according to the present invention. Further embodiments and many of the advantages mentioned above may result from the drawings. The elements shown in the drawings are not necessarily drawn to scale.

In the Figures of the drawing, identical elements, features and components, which are functionally identical and have the same effect, are each indicated by the same reference signs, unless otherwise specified.

FIG. 1 shows a schematic side view of an embodiment of a cold air therapy device 1. The cold air therapy device 1 shown in FIG. 1 comprises a cooling device 2, an air guiding device 3, a cold air outlet 4 and an air disinfection device 5. The air guiding device 3 couples the cooling device 2 to the cold air outlet 4. The air disinfection device 5 is arranged directly at the cold air outlet 4 and is integrally coupled to it. The shape of the air disinfection device 5 is configured to correspond to the cross-section of the air guiding device 2.

The air guiding device 3 enables cooled air to be directed from the cooling device 2 to the cold air outlet 4 in the form of an air flow. By means of the cold air outlet 4, the cooled air flow directed from the cooling device 2 via the air guiding device 3 may be applied to a body surface to be cooled. The air disinfection device 5 reduces the germ load and/or bacterial load of the air cooled by the cooling device 2, which air flows through the air guiding device 3 to the cold air outlet 4.

The air disinfection device 5 is shown schematically in FIG. 1 to be arranged between the air guiding device 3 and the cold air outlet 4. It is of great importance that the air flow through the air guiding device 3 will not be impaired excessively by the air disinfection device 5. In order to ensure a highly efficient reduction of the germ load in the air, it is preferable that the air flows substantially smooth and without any turbulence past the air disinfection device 5.

Preferably, the air guiding device 3 is formed as a hose manufactured from a flexible, airtight material, for example plastic.

With respect to the configuration shown in FIG. 1, it is preferable that the air flow may be controlled safely, since only the transition from the air guiding device 2 to the air disinfection device 5 must be considered for and not any other transition from the air disinfection device 5 back to the air guiding device 3.

According to this embodiment, it is also preferable that the air disinfection device 5 is easy to maintain and/or to replace in the event of a malfunction, as the air disinfection device 5 may be accessed easily.

FIG. 2 shows a schematic sectional view of an air disinfection device 5. The air disinfection device 5 comprises a housing 6 and a UV light source 7. The housing 6 of the air disinfection device 5 comprises an inlet region 8 and an outlet region 9.

The UV light source 7 is arranged centrally in relation to a direction of air flow. The germ load and/or the bacterial load of the air flowing through the housing 6 is reduced by the UV light which is emitted by the UV light source 7.

The effect of the air disinfection device 5 shown in FIG. 2 depends on the radiation power acting on an air volume flowing through the air disinfection device 5. The percentage of germs, bacteria or the like inactivated by the UV light is determined by the radiation dose absorbed by the germs, bacteria or the like. The greater the radiation dose absorbed by the germs, bacteria or the like, the greater the percentage of germs, bacteria or the like inactivated by the UV light. The radiation dose absorbed by the germs, bacteria or the like results on the one hand from the radiation power generated by the at least one UV light source, and on the other hand from the dwell time of the germs, bacteria or the like in the air disinfection device in which they are exposed to the UV light. The dwell time of the germs, bacteria or the like in the air disinfection device results in turn from the dimensions and geometry of the air disinfection device and the flow velocity of the air flow. At the same time, it should also be ensured that all air flowing into the air disinfection device 5 remains inside the air disinfection device 5 for a sufficient amount of time. Flow turbulences caused by the configuration of the housing 6 or the arrangement of the UV light source 7 should therefore be prevented if possible. In addition to the configuration shown in FIG. 2, there is a large number of optional configurations for the air disinfection device with which a reduction of the germ load and/or the bacterial load of the air flow by, for example, at least 50%, preferably by at least 90% and, particularly preferred, by at least 99% may be achieved.

In the embodiment shown in FIG. 2, the air flows in a straight laminar flow through the housing 6. A laminar flow shows no turbulence, which is why the air flowing through the housing 6 is uniformly irradiated. In addition, the housing 6 may easily be integrated into the air guiding device 3 in a straight line, since the direction of the air flow does not change at the transition between the air guiding device 3 and the housing 6.

FIG. 3 shows a schematic sectional view of another air disinfection device 5, which comprises a housing 6 and a UV light source 7. The housing 6 of the air disinfection device 5 comprises an inlet region 8 and an outlet region 9.

In the embodiment shown in FIG. 3, the inlet region 8 and the outlet region 9 are arranged in such a way that the air flows through the housing 6 along a spiral path around the UV light source 7. This increases the time that the air remains in the housing 6, which also means that more UV light acts on the air, resulting in an increased reduction in the bacterial load of the air.

In the embodiment shown in FIG. 3, the outlet region 9 is configured to have a funnel-shaped configuration, and it is preferred that the outlet region 9 is adapted to prevent any turbulences of the air flow in the outlet region 9.

FIG. 4 shows a schematic sectional view of another air disinfection device 5. The configuration of the air disinfection device 5 shown in FIG. 4 comprises a housing 6 and a ring-shaped UV light source 7 which is accommodated in the housing 6. The ring-shaped UV light source 7 comprises a tube 10 of transparent material through which air may flow from an inlet region 8 to an outlet region 9.

The tube 10 separates the UV light source 7 from the air flow, which is therefore not affected by the UV light source 7, without preventing the disinfecting effect of the UV light of the UV light source 7 on the air flow.

FIG. 5 shows a schematic sectional view of a further air disinfection device 5. The configuration of the air disinfection device 5 shown in FIG. 5 comprises a housing 6 and three UV light sources 7, which are accommodated within the housing 6 in such a way that an air flow flows on its way from an inlet region 8 of the housing 6 to an outlet region 9 of the housing 6 along a meandering path between the UV light sources 7.

According to the exemplary embodiment shown in FIG. 5, it takes a relatively long time for an amount of air to pass through the air disinfection device 5, while the amount of air is—at the same time—exposed to the radiation power of several UV light sources 6. In this way, it is preferred that the disinfection effect of the air disinfection device 5 may be increased.

In the embodiments shown so far, it has not been explicitly shown how the air disinfection device 5, in particular the housing 6 or the tube 10 thereof, is inserted into the flow path of the air flow directed towards the body region, for example within an air guiding device 3. It is preferred that the housing 6, or the tube 10, respectively, is formed to correspond to the flow path of the air flow, as defined by the cross-section of the air guiding device 3, for example. By adapting the housing to the cross-section of the air guiding device, any turbulence in the air flow may be reduced. It is preferred that a uniform irradiation acting on the air flow is achieved by a turbulence-free air flow.

FIG. 5 shows a tube 10 made of transparent material. It is also conceivable to provide individual elements of transparent material, such as flat or curved plates, to separate the UV light source from the air flow to be applied to the body surface. Transparent materials such as glass or plastic, e.g. polymethylmethacrylate, may be used.

The UV light sources 7 shown so far may preferably be configured as low-pressure mercury vapor lamps, which have a high efficiency and output, at comparatively low cost. The advantageously high intensity of the UV light emitted by low-pressure mercury vapor lamps results in a correspondingly high radiation dose absorbed by the air flowing through them.

Alternatively, the UV light sources 7 may also be configured as LEDs or lasers. LEDs have an advantageously small size and may therefore be mounted in a variety of ways, allowing more flexible configurations of the air disinfection device 5. Several UV light sources 7 may also be provided, as well as in any combination of the above-mentioned embodiments.

FIG. 6 shows a schematic side view of another exemplary embodiment of a cold air therapy device 1. The cold air therapy device 1 shown in FIG. 6 comprises a device housing 11 in which a cooling device 2, a ventilation device 12 and an air filter 13 are accommodated.

The ventilation device 12 generates an air flow, by means of which ambient air is directed from outside the device housing 11 through the air filter 13 and the ventilation device 12 to the cooling device 2. From the cooling device 2, the air flow which is now cooled down is directed through an air guiding device 3 to a cold air outlet 4, by means of which the cooled air flow may be applied to a body region.

With reference to FIG. 6 a number of possible positions 14 for an air disinfection device, which is not shown in FIG. 6, are also indicated. An air disinfection device may be positioned outside the device housing 11 at a position where ambient air is drawn in by the ventilation device 12. An air disinfection device may also be positioned in the direction of the air flow directly before or directly after the air filter 13, between the ventilation device 12 and the cooling device 2, or inside or outside the device housing 11 at a position where the air flow is directed into the air guiding device 3. The air disinfection device may also be integral with the cooling device 2, the air guiding device 3 or the cold air outlet 4.

Each of the positions 14 shown in FIG. 6 for an air disinfection device has its own advantages. The closer the air disinfection device is positioned to the cold air outlet 4, the lower the probability that the air flow will be contaminated again after the air flow has passed the air disinfection device. Accommodation of the air disinfection device in or on the device housing 11 allows the provision of a larger and generally more efficient air disinfection device, which enables more effective disinfection of the air flow. Depending on the positioning of the air disinfection device, maintenance work may be performed more easily.

For the sake of simplicity, the air guiding device 3 is shown in FIG. 6 as a straight, rigid tube. A rigid tube offers the advantage that an air disinfection device may be integrated into the tube particularly easily. It is also conceivable that the air guiding device 3 is configured—in further embodiments—as a flexible hose made of plastic, for example, which makes the air guiding device easier to handle.

Even though only one exemplary embodiment of an air disinfection device has been used to explain the principles of the present invention, it is of course also conceivable to provide a number of air disinfection devices in a cold air therapy device 1, wherein the air disinfection devices may be configured similarly of differently.

FIG. 7 shows a schematic cross-sectional view in the direction of flow of an air disinfection device 5, which comprises a housing 6 and five UV light sources 7 in total. The housing 6 comprises a circular cross-section and is covered with a UV light reflecting material 15. The five UV light sources 7 are arranged in the form of a pentagon in a central area in an almost circular shape with respect to the cross-section of the housing 6.

Air flowing through the housing 6 flows between the ring of UV light sources 7 and the wall of housing 6. In this configuration, the air flow is only slightly obstructed by the UV light sources 7 such that creation of undesirable turbulence is avoided. In addition, the UV light emitted by the UV light sources 7 is reflected by the UV light reflecting material 15, which advantageously increases the effective intensity of the UV light acting on the air, and thus increases the efficiency of the disinfection.

Alternatively to the arrangement shown in FIG. 7, the UV light sources 7 may also be arranged along the circumference of the housing 6. In this way a higher radiation intensity may be achieved in the outer areas of the air flow. It is also conceivable to mount the UV light sources 7 adjacently arranged to each other, which allows an advantageously compact design of the air disinfection device 5.

Preferably, the UV light reflecting material 15 may comprise aluminum, particularly aluminum foil, polytetrafluoroethylene, particularly in the form of a foil, and/or polycarbonate. The housing 6, for example, may be made of aluminum, which simplifies the manufacturing of the air disinfection device 5. Polytetrafluoroethylene has an advantageously high reflection factor of at least 95%. It is relatively inexpensive to coat the housing 6 with aluminum foil. Using polycarbonate as a reflective material is also inexpensive and easy to produce by means of injection molding.

### LIST OF REFERENCE SIGNS

1 Cold air therapy device
2 Cooling device
3 Air guiding device
4 Cold air outlet
5 Air disinfection device
6 Housing
7 UV light source
8 Inlet region
9 Outlet region
10 Tube
11 Device housing
12 Ventilation device
13 Air filter
14 Position
15 UV light reflecting material

1. Cold air therapy device (1) for applying a cooled air flow to a body surface, comprising:
a cooling device (2) which is configured to cool the air flow to be applied to the body surface;
an air guiding device (3) which is coupled to the cooling device (2) and which is configured in such a way as to direct the air flow cooled by the cooling device (2) and to be applied to the body surface to a cold air outlet (4); and
an air disinfection device (5) which is configured to at least reduce the germ load and/or the bacterial load of the air flow to be applied to the body surface.

2. Cold air therapy device (1) according to claim 1,
wherein the air disinfection device (5) comprises at least one UV light source (7).

3. Cold air therapy device (1) according to claim 2,
wherein the air disinfection device (5) comprises a housing (6) and is arranged in such a way that, during operation, the air flow to be applied to the body surface flows through it, wherein the UV light source (7) is arranged inside the housing (6).

4. Cold air therapy device (1) according to claim 3,
wherein the housing (6) of the air disinfection device (5) comprises a funnel-shaped outlet region (9).

5. Cold air therapy device (1) according to claim 3,
wherein the at least one UV light source (7) is arranged in a central region of the housing (6) of the air disinfection device (5) with respect to a flow direction of the air flow to be applied to the body surface.

6. Cold air therapy device (1) according to any one of claims 2 to 4,
wherein the air disinfection device (1) comprises a tube (10) made of transparent material through which the air flow to be applied to the body surface flows, and wherein the at least one UV light source (7) is arranged outside the tube (10).

7. Cold air therapy device (1) according to any one of claims 2 to 6,
wherein the air disinfection device (5) comprises a plurality of UV light sources (7).

8. Cold air therapy device (1) according to claim 7,
wherein the UV light sources (7) are arranged in a ring shape.

9. Cold air therapy device (1) according to any one of claims 2 to 8,
wherein the at least one UV light source (7) is arranged such that the air flow to be applied to the body surface flows through the air disinfection device (5) along a meandering or spiral path.

10. Cold air therapy device (1) according to any one of claims 2 to 9,
wherein at least one interior surface of the housing (6) of the air disinfection device (5) is covered or coated with a UV light-reflecting material (15), preferably with aluminum.

11. Cold air therapy device (1) according to any one of the preceding claims,
wherein the air disinfection device (5) is arranged at the cold air outlet (4), and is particularly formed integrally with the cold air outlet (4).

12. Cold air therapy device (1) according to any one of the preceding claims, further comprising an air filter (13), which is preferably arranged behind the air disinfection device (5) with respect to the flow direction of the air flow to be applied to the body surface.

13. Cold air therapy device (1) according to any one of the preceding claims, wherein a ventilation device (12) is provided which is configured to generate and/or accelerate the air flow to be applied to the body surface.

14. Method of applying a cooled air flow to a body surface, in particular by means of a cold air therapy device according to one of the preceding claims, comprising the steps of:
cooling the air flow to be applied to the body surface;
directing the cooled air flow to be applied to the body surface to the body surface; and
reducing the germ load and/or the bacterial load of the air flow to be applied to the body surface.

15. Use of an air disinfection device for reducing the germ load and/or the bacterial load of an air flow of a cold air therapy device to be applied to a body surface before its application to the body surface.
